(19) United States
(12) Patent Application Publication
(10) Pub. No.: US 2020/0288780 A1
(43) Pub. Date: Sep. 17, 2020

(54) SMOKING VAPORIZER WITH BUILT IN SMOKE FILTRATION SYSTEM

(71) Applicants: Hunter Martin, Liberty Township, OH (US); Chase Martin, Liberty Township, OH (US)

(72) Inventors: Hunter Martin, Liberty Township, OH (US); Chase Martin, Liberty Township, OH (US)

(21) Appl. No.: 16/816,406

(22) Filed: Mar. 12, 2020

Related U.S. Application Data
(60) Provisional application No. 62/818,060, filed on Mar. 13, 2019.

(51) Int. Cl. A24F 40/485; A24F 13/00; A24F 40/20; A24F 40/10

(52) U.S. Cl. CPC A24F 40/485; A24F 13/00; A61M 15/06; A24F 40/10; A24F 40/20

(57) ABSTRACT

A vapor dispenser includes a bifurcated housing having a body attached to a mouthpiece. A vaporizer cavity of the bifurcated body pneumatically communicates with an inlet orifice of the mouthpiece. A vaporizing component is received in the vaporizing cavity. An exhaust cavity of the bifurcated body pneumatically communicates with an outlet orifice of the mouthpiece. The exhaust cavity is pneumatically separated from the vaporizer cavity. A one-way valve is positioned in a selected one of: (i) the exhaust cavity; and (ii) outlet orifice and that: (i) opens in response to exhalation through the bifurcated mouthpiece; and (ii) closes in response to inhalation through the bifurcated mouthpiece. An air filter is received in the exhaust cavity.

### CROSS-REFERENCE TO RELATED APPLICATIONS

This application claims the benefit of priority under 35 U.S.C. § 119(e) to U.S. Patent Application Provisional Application Ser. No. 62/818,060, entitled “SMOKING VAPORIZER WITH BUILT IN SMOKE FILTRATION SYSTEM”, filed Mar. 13, 2019, the contents of which are hereby incorporated by reference in their entirety for any purpose.

### BACKGROUND

### 1. Technical Field

The present disclosure generally relates to personal vaporizer devices, and more specifically to personal vaporizer devices that filter first-hand escalations and second-hand vapors.

### 2. Description of the Related Art

Electronic cigarettes (eCigs) that vaporize a liquid for human inhalation are becoming increasingly used for ingesting nicotine, flavors and marijuana. Many types of electronic vaporizers exist on the market, such as electronic cigarette vaporizers (a/k/a E-cigarettes vaporizers or E-cigarettes), which simulate the feeling of smoking, but without burning the tobacco or other contents. Thus, the E-cigarette does not generate smoke, but rather typically employs a heating element that atomizes a liquid solution or other contents to produce an aerosol or vapor. The user thus inhales this vapor rather than inhaling smoke. Such is commonly referred to as “vaping”, which has certain advantages over smoking. For example, inhaling a vapor or aerosol (e.g., vaping) containing an active ingredient (e.g., nicotine) is believed to be a safer alternative to inhaling smoke. As another example, vaping can provide a more efficient delivery of the active ingredient to the user, since pyrolysis (or another type of thermochemical decomposition) can destroy a significant portion of the active ingredient.

Electronic vaporizers or “E-vapes” are typically designed to operate with specific types of materials. For example, some E-vapes are configured to operate with liquids or oils, which can contain the active ingredients. Other E-vapes are configured to operate with plant products. Still other E-vapes are configured to operate with other materials such as a wax or wax-like material containing the active ingredient.

The present invention relates generally to a medical cannabis delivery device such as but not limited to inhaling and exhaling the vapor produced by an electric device referred to such as but not limited to a vaporizer, atomizer, cartomizer, or a clearomizer. If one vaporizes a piece of medical cannabis wax it is called an atomizer; or if an oil saturated polyfilled material is wrapped around a heating source then it's a cartomizer; or if oil is dripped from a separate holding tank using a silica wick then it's a clearomizer.

The vaporizing or aerosolization of the liquid can be carefully controlled for each dispensing. Even with this control, electronic smoking devices do still cause emissions, which may potentially pose a health and/or environmental risk, and may be viewed negatively in a public or social environment.

### BRIEF SUMMARY

In one aspect, the present disclosure provides a vapor dispenser that includes a bifurcated housing having a body attached to a mouthpiece. A vaporizer cavity of the bifurcated body pneumatically communicates with an inlet orifice of the mouthpiece. A vaporizing component is received in the vaporizing cavity. An exhaust cavity of the bifurcated body pneumatically communicates with an outlet orifice of the mouthpiece. The exhaust cavity is pneumatically separated from the vaporizer cavity. A one-way valve is positioned in a selected one of: (i) the exhaust cavity; and (ii) outlet orifice and that: (i) opens in response to exhalation through the bifurcated mouthpiece; and (ii) closes in response to inhalation through the bifurcated mouthpiece. An air filter is received in the exhaust cavity.

The smokeable materials herein may include, but are not limited to, tobacco, botanicals (e.g., cannabis, chamomile), pharmaceuticals, nutraceuticals, natural or artificial flavorants, coffee grounds or coffee beans, mint, lemon, honey, tea leaves, cocoa, or any other substance providing a benefit or sensation to an end user.

The smokeable materials herein may be provided in loose leaf form, cut form, shredded form, chopped form, packed form, or any other natural or processed form. As described elsewhere herein, in some examples, the smokeable material may comprise fine pieces of tobacco. In other examples, the smokeable material may comprise loose leaf tobacco. In yet other examples, the smokeable material may comprise loose leaf, shredded or chopped botanicals (e.g., loose leaves, shredded. The smokeable material comprise a vapor forming medium (e.g., glycerin).

Active elements contained in botanicals may vaporize at different temperatures. The device may be calibrated to establish a single stable temperature, intended for vaporizing solely tobacco or solely chamomile, for example. A control means may be used to select a variety of temperature settings. The user may choose which setting based on the type of cartridge used. The control means can effect a desired temperature mechanically, such as by changing flow rate of the valve, or electronically, such as by electromechanical valve and micro-controller intermediary.

In accordance with an aspect of this disclosure, there is provided a vaporization device for vaporizing phyto material and/or phyto material extracts. The vaporization device can include a vaporization element that is usable to heat phyto material extracts to a desired vaporization temperature to generate an extract vapor. The vaporization device can define a fluid pathway extending from the vaporization element to an inhalation aperture. The extract vapor can flow through the fluid pathway to the inhalation aperture whereby a user can inhale the vapor. The vapor may be drawn into and through the fluid pathway by a user inhaling via the inhalation aperture.

One or more embodiments described herein relate generally to vaporization of phyto material and phyto material products. Phyto material products maybe derived from phyto materials such as the leaves or buds of cannabis plants. Derived phyto material products may be referred to by various terms, such as oils, extracts, concentrates, tinctures etc.

For simplicity and clarity, unless otherwise specified, the terms “vaporizing phyto material” or “vaporization of phyto material” (and variants thereof) are used herein as general terms to encompass the vaporization of phyto materials such as leaves or buds as well as the vaporization of derived phyto material products such as extracts.

Phyto material extracts (oils, extracts, concentrates, tinctures etc.) can be derived from phyto materials such as the leaves or buds of cannabis plants. Typically, phyto materials may be leafy while phyto material extracts may have an oily or waxy consistency. These phyto material extracts may be in liquid and/or solid states. Heat can be applied to these phyto material extracts to cause them to boil and/or sublimate and release a vapor.

In some cases, the phyto material products may be derived from plants such as cannabis plants using various processing techniques, which can include additives in the derived products. In other cases, they may be extracted directly e.g. from oils or resins secreted by, or excreted from, plants such as cannabis plants without additional additives.

Various phyto material products derived from plant matter can be vaporized for aromatherapy or therapeutic purposes. For instance, phyto material extracts derived from parts of the cannabis plants, such as the buds and/or leaves, may be vaporized. A user may inhale the cannabis vapor to achieve associated therapeutic effects.

Various methods of vaporizing phyto materials, such as cannabis products, are known. For cannabis oils or extracts, temperatures in the range of about 500 to 700 degrees Fahrenheit may be applied to vaporize these oils or extracts. In many cases, a metal or ceramic heating element may be heated using a torch in order to reach the desired temperature. The heated heating element may then be brought into contact with the extract to generate vapor. This vapor can then be inhaled by a user, sometimes after passing through a cooling channel.

The instant disclosure teaches refillable cartridges and disposable exhalation cartridges for use with and in a heater base wherein the cartridge contains the organic material to be vaporized and the exhalation filter provides for an air filter for filtering exhaled vapor and air.

In general, a vaporization device in accordance with embodiments described herein includes a vaporization element. The vaporization element can be used to vaporize phyto material and/or phyto material extract to generate vapor that can be inhaled by a user.

The vaporization element typically includes a heating element with an extract holder portion or phyto material contact element that can be configured to receive phyto material extract. Phyto material extract that is to be vaporized can be positioned in the extract holder portion (e.g. on a phyto material contact surface of the phyto material contact element) to be vaporized.

A heater can be positioned proximate to the extract holder portion. For instance, the heater may be formed as part of the heating element and may be positioned adjacent to, or even as part of (e.g. partially embedded or sintered into), the phyto material contact element. The heater can be configured to heat the phyto material contact element (and thus the phyto material contact surface) to a predefined vaporization temperature. The predefined vaporization temperature can be selected as temperature suitable for boiling the phyto material or phyto material extract to generate a vapor. The predefined vaporization temperature can be selected to vaporize the phyto material without causing the phyto material or extract to combust.

In embodiments described herein, the heater may be an electric heater. For instance, the electric heater may include a resistive heater that generates heat as current flows therethrough. The temperature of the heater may be adjustable to provide a desired vaporization temperature, e.g. by adjusting the level of current flowing through the resistive heater.

The vaporization device also generally includes a fluid pathway that extends from a vapor inlet to an inhalation aperture. The vapor inlet can be positioned proximate to the heating element. In particular, the vapor inlet may be positioned proximate to, and in fluid communication with, the phyto material contact surface. The vapor inlet may be positioned to capture some or all of the vapor released when the phyto material extract is vaporized. The inhalation aperture can be used by an individual to inhale the vapor received from the vapor inlet which can be drawn through the fluid pathway.

The fluid pathway may include one or more intermediate portions between the vapor inlet and the inhalation aperture. For instance, the fluid pathway may include a filtering section and/or a cooling section. The filtering section may filter the vapor (and ambient air) passing through the fluid pathway before it reaches the inhalation aperture. Filtering the vapor may remove particulate matter that was entrained with the vapor as it entered the vapor inlet.

The fluid pathway may also include an additional cooling section. The cooling section generally refers to a portion of the fluid pathway providing heat exchange between the fluid (i.e. vapor and ambient air) in the fluid pathway and other fluids (such as ambient air or water) adjacent to, or positioned within, the fluid pathway. The cooling section may reduce the temperature of the vapor to a temperature more suitable for inhalation.

In some cases, a combined filtering and cooling section may be provided. For example, the vaporization device may include a water trap positioned in the fluid pathway between the vapor inlet and the inhalation aperture. The water trap may remove particulate matter from the vapor and air passing through the fluid pathway. The water trap can also cool the vapor passing therethrough.

In some embodiments, the filtering and/or cooling sections may be provided by a separate vapor processing device. The vapor processing device can be coupled to the vaporization element.

In some embodiments, the vaporization element may include filtering and/or cooling sections. For instance, the vaporization element may include an elongated member defining the fluid pathway. The elongated member may cool the vapor as it passes therethrough because of heat transfer between the vapor and ambient air around the elongated member. In some cases, a filtering component such as a screen may be placed in the fluid pathway to filter vapor passing therethrough.

In some embodiments, the vaporization device may also include various power and/or control components. For example, the vaporization device may include an onboard power source. The power source may include one or more batteries. The onboard power source may be used to provide current for the electric heating element. The onboard power source may also power other electric and/or electronic components of the vaporization device, such as control circuitry that may be included in the vaporization device. In some cases, the onboard power source and/or electronic components may be provided as part of a vaporization device support unit.

The above summary contains simplifications, generalizations and omissions of detail and is not intended as a comprehensive description of the claimed subject matter but, rather, is intended to provide a brief overview of some of the functionality associated therewith. Other systems, methods, functionality, features and advantages of the claimed subject matter will be or will become apparent to one with skill in the art upon examination of the following figures and detailed written description.

### BRIEF DESCRIPTION OF THE DRAWINGS

The description of the illustrative embodiments can be read in conjunction with the accompanying figures. It will be appreciated that for simplicity and clarity of illustration, elements illustrated in the figures have not necessarily been drawn to scale. For example, the dimensions of some of the elements are exaggerated relative to other elements. Embodiments incorporating teachings of the present disclosure are shown and described with respect to the figures presented herein, in which:

FIG. 1 is side diagrammatic view illustrating an example vapor dispenser with attachable filter canister during inhalation by a user, according to one or more embodiments;

FIG. 2 is side diagrammatic view illustrating the vapor dispenser of FIG. 1 during exhalation by the user, according to one or more embodiments;

FIG. 3 is an isometric view of an example vapor dispenser having a filter door to receive a cylindrical air filter into an inside-out filter compartment, according to one or more embodiments;

FIG. 4 is a side diagrammatic view of the example vapor dispenser of FIG. 3 during inhalation, according to one or more embodiments;

FIG. 5 is a side diagrammatic view of the example vapor dispenser of FIG. 3 during exhalation, according to one or more embodiments;

FIG. 6 is an isometric view of an example vapor dispenser having a filter door to receive a cylindrical air filter into an outside-in filter compartment, according to one or more embodiments;

FIG. 7 is a side diagrammatic view of the example vapor dispenser of FIG. 6 during inhalation, according to one or more embodiments;

FIG. 8 is a side diagrammatic view of the example vapor dispenser of FIG. 6 during exhalation, according to one or more embodiments;

FIG. 9 is an isometric view of an example vapor dispenser having replaceable planar air filters, according to one or more embodiments;

FIG. 10 is an isometric view of an example vapor dispenser having a three-chamber barrel, according to one or more embodiments;

FIG. 11 is a top view of the three chamber barrel of FIG. 10, according to one or more embodiments; and

FIG. 12 is an isometric view of an example refillable cartridge of the example vapor dispenser of FIG. 10, according to one or more embodiments.

### DETAILED DESCRIPTION

The present innovation provides a vapor dispenser that provides a smoking vaporizer with built-in smoke filtration system. This innovation combines the smoking vaporizer and an air filter into a single unit. In one or more embodiments, a smoking vaporizer for marijuana (e.g., cannabis flower or cannabis oil) with an air filter that allows the user to “take a hit” from the vaporizer and exhale the smoke into a filter all in one smooth action.

Second hand smoke and the smells associated with the use of medical and recreational marijuana is highly offensive. Because the user's lips never leave the pen, nearly all smell and smoke are eliminated, and the user can smoke in a discreet and inoffensive manner. Filter paper such as P100 Organic vapor paper can be used to filter 100% of the smoke. In one or more embodiments, simple and cost-effective filters can be used such as high efficiency particulate air (HEPA) filters with activated charcoal.

A method, system and device is disclosed which can at least one of reduce the odor in and about a heating chamber in a vaporizer and reduce the odor of exhaled breath.

Electronic vaporizers are typically designed to operate with specific types of vaping contents in connection with delivery of the active ingredients. As used herein, “vaping contents” is intended to refer to the contents or materials that contain or convey the active ingredient(s) and are exposed to heating from a heating element of the host e-vape device. Such vaping contents can be liquids or oils, plant products, or a wax or wax-like material. However, a particular E-vape device is typically designed to operate with one, specific type of vaping contents. For example, an E-vape device designed to vaporize a plant product typically will not correctly operate with a liquid product used by different E-vape devices, and vice versa.

In some embodiments, the disclosed subject matter relates to an electronic vaporizer device that can support different types of vaping contents. Such can be accomplished in a variety of ways. For example, in some embodiments, the E-vape device can comprise a single base power unit (e.g., comprising the battery or other power source that powers the heating elements) and an interchangeable vapor delivery system portion (e.g., comprising the heating chamber where the vaping contents are exposed to the heating elements). In one or more embodiments, a single power unit base can operate many different types of vapor delivery systems, such as a vapor delivery system that is specifically designed for liquid vaping contents, a second vapor delivery system that is specifically designed for plant material vaping contents, or others. Since different vaping contents can have different temperature requirements (e.g., to boil or vaporize the active ingredient without burning the vaping contents or active ingredient), in some embodiments, the vapor delivery system can determine the heat that will be produced by the heating elements. Such can be configurable or can be determined by the type of vaping contents for which the particular vapor delivery system is designed.

In one or more embodiments, the disclosed subject matter further relates to a sealed capsule, which can also be referred to as a pod or sealed pod. This sealed capsule can comprise the vaping contents and can be configured to have specific dimensions to fit an associated vaping chamber. As the name implies, the sealed capsule can be sealed to prevent the vaping contents from escaping, or mitigate such to a substantial degree. In the case of a plant material type of vaping contents, the sealed capsule can be formed in a mesh arrangement or another suitable arrangement to prevent or mitigate the escape of plant material. In the case of a liquid type of vaping contents, the sealed capsule can be spill-proof, or in some embodiments, hermetically sealed.

In one or more embodiments, the sealed capsule can contain vaping contents representing a single-use. In some embodiments, the sealed capsule can contain vaping contents representing a single dose of the active ingredient, which can be determined according to a defined scale.

In one or more embodiments, the sealed capsule can be of a standardized size that is supported by the E-vape device. It is understood that the E-vape device can thus support substantially any type vaping contents (e.g., liquids, solids, fibrous plant material, etc.) by having a chamber that supports the sealed capsule. In some embodiments, the E-vape device can have a configurable or controllable heating element. Such can be employed to match the temperature produced by the heating element to the vaping contents. In some embodiments, the sealed capsule can include an indicator of a suggested setting for the E-vape device and/or the heating elements of the E-vape device.

A vaporizer system, device and method which provides for heating of a cartridge or a section thereof is disclosed. In one or more embodiments, cartridges are disposable but in some embodiments may be refillable. Vaporizing plant material for inhalation of plant borne chemicals is considered by some to be less harmful then combusting the plant material. Tobacco and cannabis are examples of such material.

In one or more embodiments, the vaporizer systems and methods disclosed include one or more of a base having a cartridge interface to receive a disposable cartridge comprising: a heater element; an on/off switch; a battery; an illuminated indicator; a temperature sensor in proximity to the heater element; a controller in signal communication with the heater element, battery, indicator, temperature sensor and the on/off switch; a disposable cartridge; and, whereby pressing the on/off switch turns on power to the heater.

The instant disclosure teaches refillable cartridges and disposable exhalation cartridges for use with and in a heater base wherein the cartridge contains the organic material to be vaporized. Cartridges may be tubular, conical, or flat. Some cartridges are multipart. Cartridges may have sections which are formed of different materials with different properties to facilitate at least one of strength, insulation, conduction, ease of use, thermal transfer for heating, and containment for the plant material or for extract therein. In some embodiments, a cartridge is disposable having contained material for vaporization to prevent direct contact with the heater or a heater chamber thereby reducing the clogging, odiferousness, vapor condensate material or other build up within the heater or heating chamber. In some embodiments, the fluid pathway from the organic material to the inhalation point is integral to the cartridge and disposable thereby reducing the clogging, odiferousness, vapor condensate material or other build up in the fluid pathway.

In some embodiments, the heater is a single heater placed or moved into proximity with the material (in the cartridge) to vaporize wherein heat is supplied to and through the cartridge or a portion thereof. In some embodiments, a controller, such as a microprocessor with hardware and/or software logic turns on/off heating element. In some embodiments, multiple heating elements are used to form zones to heat different sections of the cartridge at different times.

The instant disclosure also teaches aspects of one or more reusable inhalation cartridges wherein the cartridge has a chamber containing organic material to be vaporized. A fluid pathway for air to pass through organic material being heated for vaporization to the inhalation point is integral to the cartridge. In some embodiments, heater elements are arrayed and the controller or controllers turn heater elements on/off to apply heat to a selected portion or portions of the cartridge at a time. In some embodiments, the cartridge is moved across a single heater to bring a portion of the cartridge into close proximity to the heating element.

In the following detailed description of exemplary embodiments of the disclosure, specific exemplary embodiments in which the disclosure may be practiced are described in sufficient detail to enable those skilled in the art to practice the disclosed embodiments. For example, specific details such as specific method orders, structures, elements, and connections have been presented herein. However, it is to be understood that the specific details presented need not be utilized to practice embodiments of the present disclosure. It is also to be understood that other embodiments may be utilized and that logical, architectural, programmatic, mechanical, electrical and other changes may be made without departing from general scope of the disclosure. The following detailed description is, therefore, not to be taken in a limiting sense, and the scope of the present disclosure is defined by the appended claims and equivalents thereof.

References within the specification to “one embodiment,” “an embodiment,” “embodiments”, or “one or more embodiments” are intended to indicate that a particular feature, structure, or characteristic described in connection with the embodiment is included in at least one embodiment of the present disclosure. The appearance of such phrases in various places within the specification are not necessarily all referring to the same embodiment, nor are separate or alternative embodiments mutually exclusive of other embodiments. Further, various features are described which may be exhibited by some embodiments and not by others. Similarly, various requirements are described which may be requirements for some embodiments but not other embodiments.

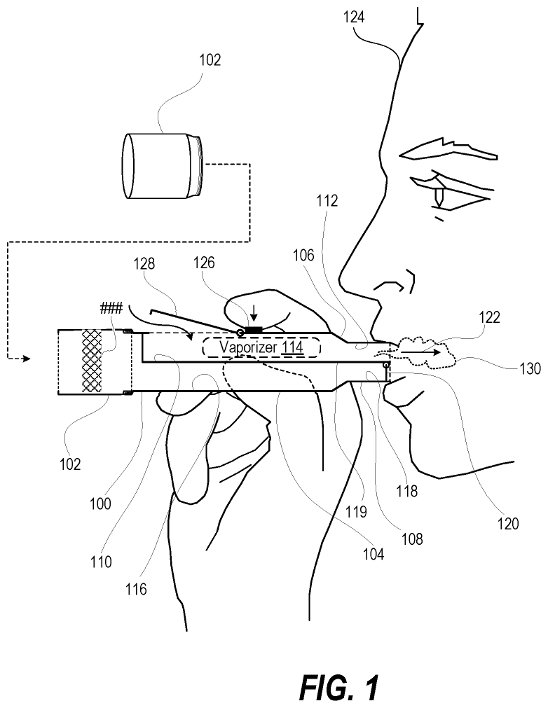

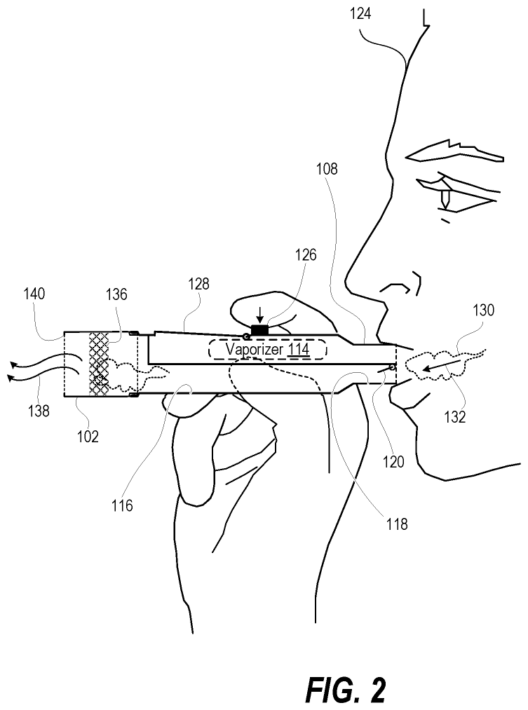

FIGS. 1-2 illustrate an example vapor dispenser 100 with an attachable air filter canister 102. With particular reference to FIG. 1, the vapor dispenser 100 includes a bifurcated housing 104 having a body 106 attached to a mouthpiece 108. A vaporizer cavity 110 of the bifurcated body 106 that pneumatically communicates with an inlet orifice 112 of the mouthpiece 108. A vaporizing component 114 is received in the vaporizing cavity 110. An exhaust cavity 116 of the bifurcated body 106 pneumatically communicates with an outlet orifice 118 of the mouthpiece. The exhaust cavity 116 is pneumatically separated from the vaporizer cavity 110 by interior wall 119. A one-way valve 120, such as a silicone mouth flap, is positioned in a selected one of: (i) the exhaust cavity 116; and (ii) the outlet orifice 118. In FIG. 1, the one-way valve 120 closes in response to inhalation 122 by a user 124 through the inlet orifice 112 of the mouthpiece 108. As the user 124 inhales, the user actuates a switch 126 that: (i) mechanically opens a vaporizer intake door 128 upstream of the vaporizing component 114 in the vaporizer cavity 110; and (ii) electrically activates the vaporizing component 114 to dispense aerosolized or vaporized product 130 such as smoke. The inhalation 122 draws the vaporized product 130 through the inlet orifice 112 into the mouth of the user 124.

FIG. 2 illustrate the user 124 releasing the switch 126, which: (i) mechanically closes the vaporizer intake door 128; and (ii) electrically deactivates the vaporizing component 114. The user 124 presents exhalation 132 of what remains of the vaporized product 130 to the mouthpiece 108. The one-way valve 120 opens in response to the exhalation 132 through the outlet orifice 118 of the mouthpiece 108. The exhalation 132 passes through the exhaust cavity 116 and into a filter cavity 134 of the attached air filter canister 102. The exhalation 132 encounters an upstream first side of air filter material 136. As the exhalation passes through from the first side to a downstream second side of the air filter material 136, organic compounds and other particulates and droplets are captured, allowing unscented air 138 to exit through an exhaust vent 140 in the air filter canister 102.

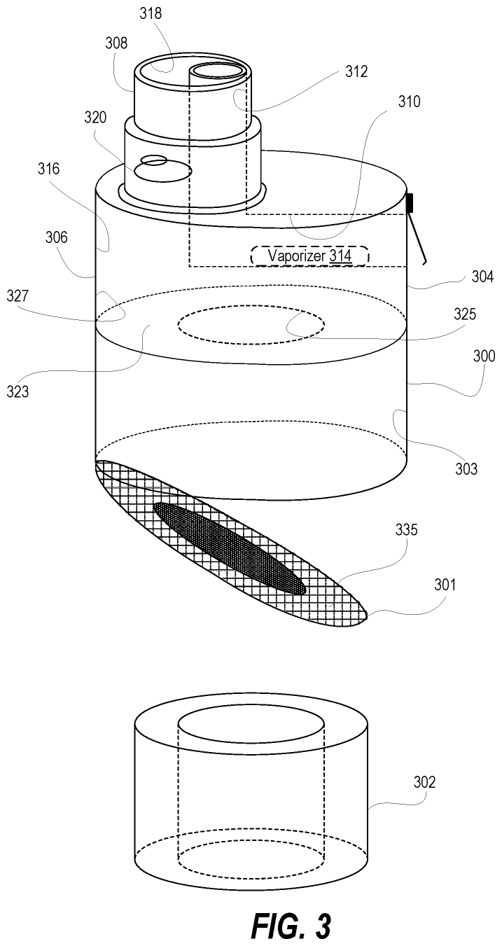

FIGS. 3-5 illustrate an example vapor dispenser 300 having a filter chamber door 301 to receive a cylindrical air filter 302 into an inside-out filter compartment 303, according to one or more embodiments. With particular reference to FIG. 3, the vapor dispenser 300 includes a bifurcated housing 304 having a bifurcated body 306 attached to a mouthpiece 308. A vaporizer cavity 310 of the bifurcated body 306 that pneumatically communicates with an inlet orifice 312 of the mouthpiece 308. A vaporizing component 314 is received in the vaporizing cavity 310. An exhaust cavity 316 of the bifurcated body 306 pneumatically communicates with an outlet orifice 318 of the mouthpiece 308. The exhaust cavity 316 is pneumatically separated from the vaporizer cavity 310 by interior wall 319. A one-way valve 320, such as a silicone mouth flap, is positioned in a selected one of: (i) the exhaust cavity 316; and (ii) the outlet orifice 318. The exhaust cavity 316 is divided by a baffle 323, which has a central opening 325, into an upper chamber 327 that communicates with the outlet orifice 312 and the inside-out filter compartment 303. The inside-out filter compartment 303 includes exhaust vents 335 that receive air passing outwardly through the cylindrical air filter 302. In one or more embodiments, the exhaust vents 335 are annularly disposed through the filter chamber door 301.

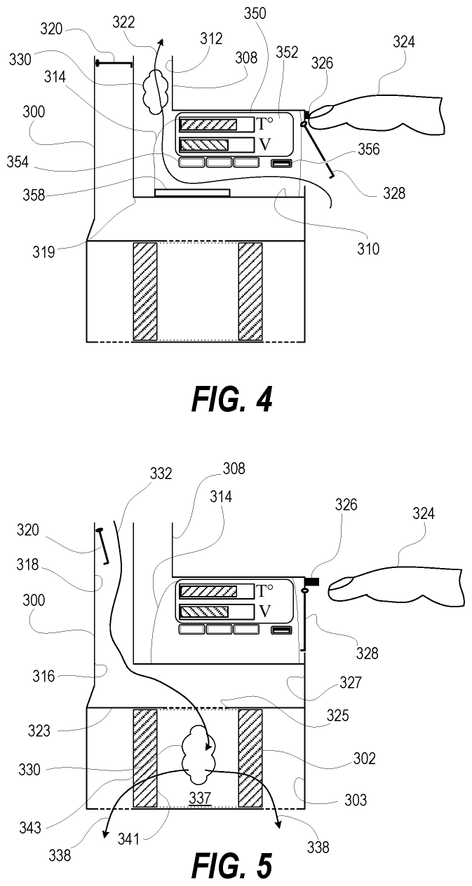

In FIG. 4, the one-way valve 320 of the vapor device 300 closes in response to inhalation 322 through the inlet orifice 312 of the mouthpiece 308. As the user 324 inhales, the user 324 actuates a switch 326 that: (i) mechanically opens a vaporizer intake door 328 upstream of the vaporizing component 314 in the vaporizer cavity 310; and (ii) electrically activates the vaporizing component 314 to dispense aerosolized or vaporized product 330 such as smoke. The inhalation 322 draws the vaporized product 330 through the inlet orifice 312.

Vaporizer component 314 can include active, electronic components including a user interface 350 that includes a display 352 for parameters such as capacity and temperature and includes user controls 354. A power interface such as a universal serial bus (USB) port 356 provides charging for user interface 350 and a heating component 358.

FIG. 5 illustrate the user releasing the switch 326 of the vapor device 300, which: (i) mechanically closes the vaporizer intake door 328; and (ii) electrically deactivates the vaporizing component 314. The user 324 presents exhalation 332 of what remains of the vaporized product 330 to the mouthpiece 308. The one-way valve 320 opens in response to the exhalation 332 through the outlet orifice 318 of the mouthpiece 308. The exhalation 332 passes through an upper chamber 327 of the exhaust cavity 316, through the central opening 325 of the baffle 323 into an interior space 337 of the cylindrical air filter 302 within the inside-out filter compartment 303. The exhalation 332 encounters an internal first side 341 of cylindrical air filter 336. As the exhalation passes through from the internal first side 341 to an external second side 343 of the cylindrical air filter 302, organic compounds and other particulates and droplets are captured, allowing unscented air 338 to exit through the exhaust vent 335 in the air filter door 331.

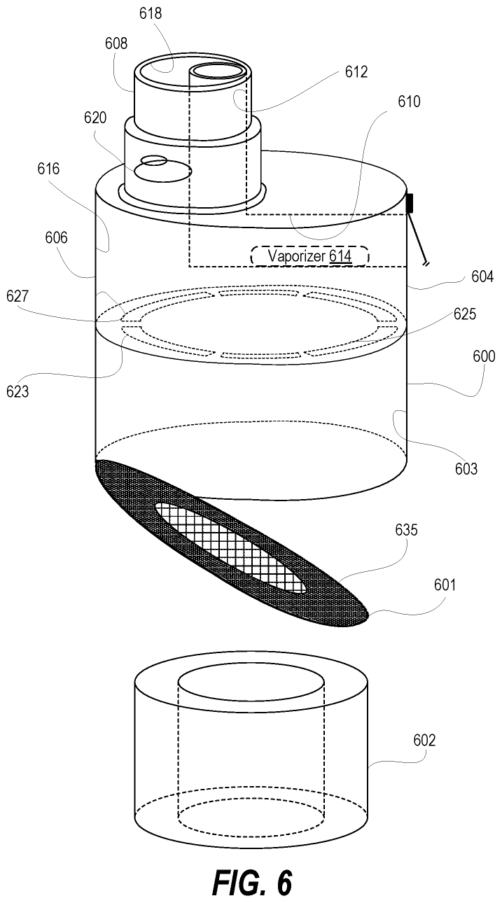

FIGS. 6-8 illustrate an example vapor dispenser 600 having a filter door 601 to receive a cylindrical air filter 602 into an outside-in filter compartment 603, according to one or more embodiments. With particular reference to FIG. 6, the vapor dispenser 600 includes a bifurcated housing 604 having a body 606 attached to a mouthpiece 608. A vaporizer cavity 610 of the bifurcated body 606 that pneumatically communicates with an inlet orifice 612 of the mouthpiece 608. A vaporizing component 614 is received in the vaporizing cavity 610. An exhaust cavity 616 of the bifurcated body 606 pneumatically communicates with an outlet orifice 618 of the mouthpiece. The exhaust cavity 616 is pneumatically separated from the vaporizer cavity 610 by interior wall 619. A one-way valve 620, such as a silicone mouth flap, is positioned in a selected one of: (i) the exhaust cavity 616; and (ii) the outlet orifice 618. The exhaust cavity 616 is divided by a baffle 623, which has one or more outer openings 625, into an upper chamber 627 that communicates with the outlet orifice 612 and the outside-in filter compartment 603. A filter chamber door 631 enables inserting a cylindrical air filter 633 into the outside-in filter compartment 603. The outside-in filter compartment 603 includes a central exhaust vent 635 that receive air passing outwardly through the cylindrical air filter 633. In one or more embodiments, the central exhaust vent 635 is centrally disposed through the filter chamber door 631.

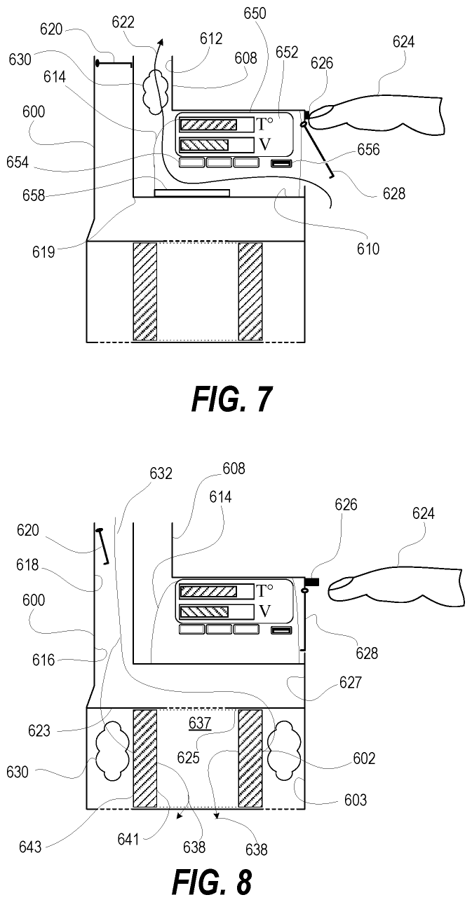

In FIG. 7, the one-way valve 620 closes in response to inhalation 622 through the inlet orifice 612 of the mouthpiece 608. As the user 624 inhales, the user 624 actuates a switch 626 that: (i) mechanically opens a vaporizer intake door 628 upstream of the vaporizing component 614 in the vaporizer cavity 610; and (ii) electrically activates the vaporizing component 614 to dispense aerosolized or vaporized product 630 such as smoke. The inhalation 622 draws the vaporized product 630 through the inlet orifice 612.

FIG. 8 illustrate the user releasing the switch 626, which: (i) mechanically closes the vaporizer intake door 628; and (ii) electrically deactivates the vaporizing component 614. The user 624 presents exhalation 632 of what remains of the vaporized product 630 to the mouthpiece 608. The one-way valve 620 opens in response to the exhalation 632 through the outlet orifice 618 of the mouthpiece 608. The exhalation 632 passes through an upper chamber 627 of the exhaust cavity 616, through the central opening 625 of the baffle 623 into an interior space 637 of the cylindrical air filter 633 within the outside-in filter compartment 603. The exhalation 632 encounters an internal first side 641 of cylindrical air filter 636. As the exhalation passes through from the internal first side 641 to an external second side 643 of the cylindrical air filter 629, organic compounds and other particulates and droplets are captured, allowing unscented air 638 to exit through the exhaust vent 635 in the air filter door 631.

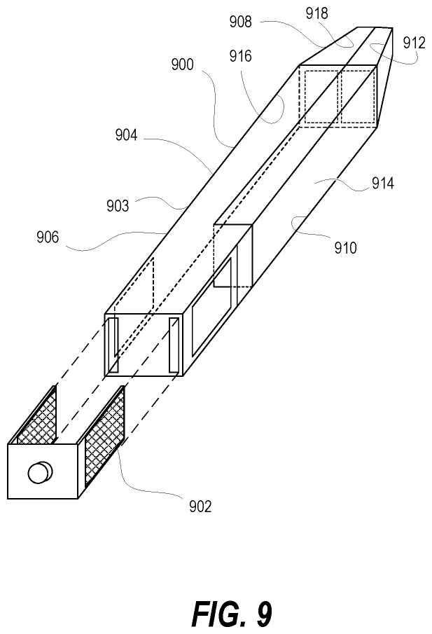

FIG. 9 illustrates an example rectangular vapor dispenser 900 having replaceable planar air filters 902. In one or more embodiments, the replaceable planar air filter 902 is part of an add-on assembly 903 to a rectangular vaporizing component 914. A bifurcated housing 904 has a body 906 attached to a mouthpiece 908. A vaporizer cavity 910 of the bifurcated body 906 that pneumatically communicates with an inlet orifice 912 of the mouthpiece 908. The rectangular vaporizing component 914 is received in the vaporizing cavity 910. An exhaust cavity 916 of the bifurcated body 906 pneumatically communicates with an outlet orifice 918 of the mouthpiece 908.

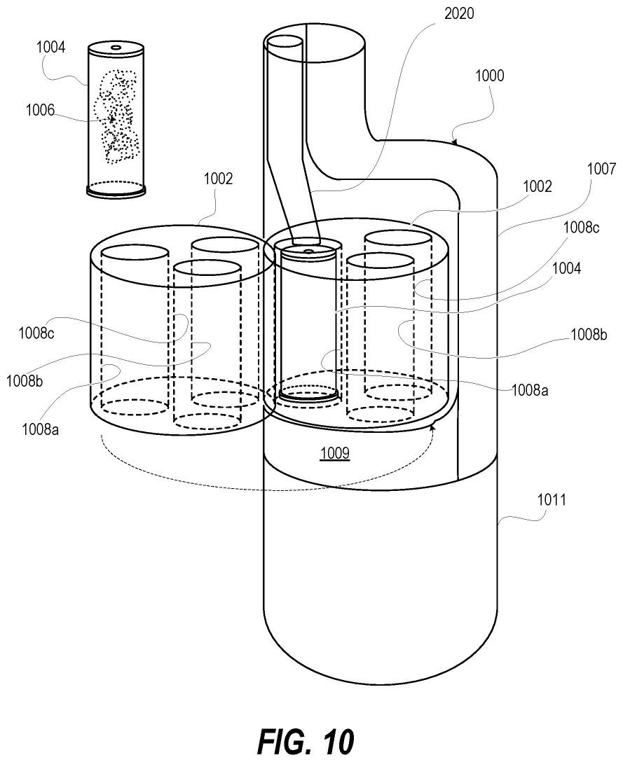

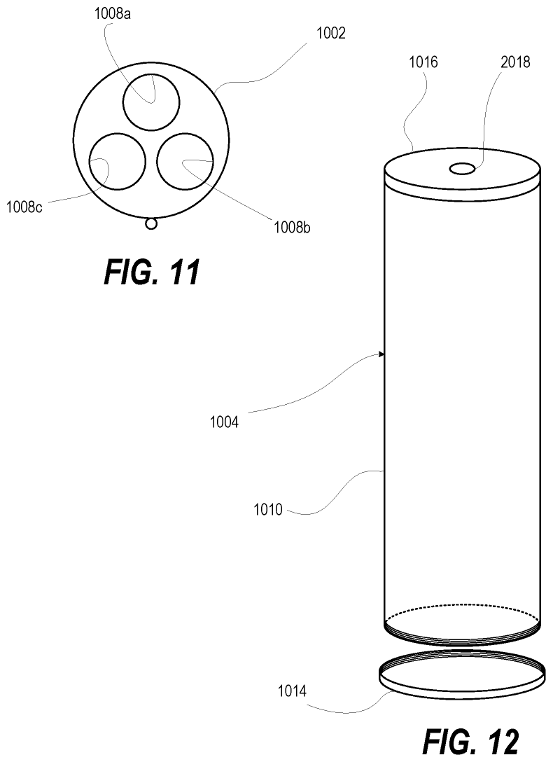

FIG. 10 illustrates an example vapor dispenser 1000 having a three-chamber barrel 1002 that receives cartridges 1004 containing vaporizing material 1006. In one or more embodiments, barrel 1002 rotates out and detaches from a bifurcated body 1007 of the vapor dispenser 1000 similar to a revolver pistol. A heating chamber 1009 adjacent to the barrel 1002 causes the vaporizing material 1006 to vaporize. Vapor dispenser 1000 includes a filter canister 1011. For clarity, FIG. 11 illustrates three chambers 1008a-1008c are radially arranged in the three-chamber barrel 1002, although other numbers of chambers may be provided, such as one, two, four or more. In one or more embodiments, FIG. 12 illustrates an example cartridge 1004 that is refillable. A steel cylinder 1010 of the example cartridge 1004 can be filled with vaporizing material 1006 such as marijuana or oils via a screw-on steel base 1014. A cap 1016 of the cartridge 1004 includes an opening 1018 that aligns with an inhalation stem 1020 of the vapor dispenser 1000 (FIG. 10).

In one or more embodiments, vapor dispenser 1000 uses a single ignition/heat system and each cartridge is rotated on top of it, just like a revolving pistol has one firing pin and barrel and each round it rotated into it and fired. The steel cartridge will be heated from the heating element and vaporize the oil or marijuana flower. When the user is done with the cartridge, the cartridge is simply rotated to the next one. The housing of the revolving component can either utilize a swing arm like a revolving pistol or the revolver can be completely built in and would be loaded by unscrewing or detaching the top portion of the pen, inserting the cartridges, and then reattaching the top of the pen. It will work just like a revolving pistol except that instead of loading in a shell filled will gunpowder, it will be filled with marijuana flower or oils. The cartridges are simply a steel tube with a specialized base and lid that allows the marijuana to be sealed until it is rotated into place and the stem of the inhalation tube meets the cartridge, whereby smoke can flow out of the cartridge and through the tube into the mouth. In one or more embodiments, instead of reusable steel cartridges that the user packs at home, disposable preloaded ones can be sold pre-packed and the user simply pops in. In one or more embodiments, the disposable ones could be made out of glass or steel.

In some embodiments, the vaporization device may include a temperature sensor in thermal communication with the heating element. The temperature sensor may be operable to measure a temperature of the heating element and to generate a temperature signal based on the measured temperature of the heating element. The control circuit can be configured to receive the temperature signal from the temperature sensor and to determine a temperature of the phyto material contact surface based on the received temperature signal.

In some embodiments, the temperature sensor may be coupled to the control circuit by the electrical connector when the electrical connector is engaged with the support unit and the electronic vaporization.

In some embodiments, the control circuit can be configured to pulse width modulate the electrical power provided to the resistive heater to maintain the phyto material contact surface at the predefined vaporization temperature.

In some embodiments, the electronic vaporization element may include a second temperature sensor and a second control circuit that is electrically coupled to the second temperature sensor and to the first control circuit. The second temperature sensor may be positioned to measure a temperature of ambient air; and the control circuit can configured to determine the predefined vaporization temperature based on the temperature of the ambient air. The control circuit may adjust the power provided to the electrical heater based on the temperature of the ambient air.

In some embodiments, the support unit may include an orientation sensor electrically coupled with the control circuit, the orientation sensor may be operable to generate a tilt signal upon determining that the support unit is not positioned in the in-use position, and the control circuit may be configured to disable the electrical heater in response to the tilt signal.

In some embodiments, the device may also include an extract ejector having an extract output port and an extract reservoir fillable with phyto material extract; and an actuator electrically coupled to the first control circuit and mechanically coupled to the extract ejector, the actuator operable to actuate the extract ejector to deposit a predefined volume of phyto material extract from the extract reservoir onto the phyto material contact surface via the extract output port.

In some embodiments, the extract ejector may be a syringe that can be filled with phyto material extract. In some embodiments, the extract ejector can include a plurality of syringes.

In some embodiments, the vaporization device may also include an ambient air input aperture upstream from the first end of the elongated member for receiving ambient air and a mass airflow meter in fluid communication with the first end of the elongated member disposed downstream of the ambient air input aperture. The mass airflow meter may measure a quantity of ambient air passing therethrough and generate initial air flow data based on an initial flow of ambient air passing therethrough. The mass airflow meter may be coupled to the control circuit, and the control circuit may process the initial air flow data and adjust at least one of the predetermined volume of the phyto material extract being deposited per unit of time onto the phyto material contact surface and the predefined vaporization temperature of the phyto material contact surface based on the initial air flow data.

While the disclosure has been described with reference to exemplary embodiments, it will be understood by those skilled in the art that various changes may be made and equivalents may be substituted for elements thereof without departing from the scope of the disclosure. In addition, many modifications may be made to adapt a particular system, device or component thereof to the teachings of the disclosure without departing from the essential scope thereof. Therefore, it is intended that the disclosure not be limited to the particular embodiments disclosed for carrying out this disclosure, but that the disclosure will include all embodiments falling within the scope of the appended claims. Moreover, the use of the terms first, second, etc. do not denote any order or importance, but rather the terms first, second, etc. are used to distinguish one element from another.

The terminology used herein is for the purpose of describing particular embodiments only and is not intended to be limiting of the disclosure. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The description of the present disclosure has been presented for purposes of illustration and description, but is not intended to be exhaustive or limited to the disclosure in the form disclosed. Many modifications and variations will be apparent to those of ordinary skill in the art without departing from the scope of the disclosure. The described embodiments were chosen and described in order to best explain the principles of the disclosure and the practical application, and to enable others of ordinary skill in the art to understand the disclosure for various embodiments with various modifications as are suited to the particular use contemplated.

What is claimed is:

1. A vapor dispenser comprising:
a bifurcated housing having a body attached to a mouthpiece;
a vaporizer cavity of the bifurcated body that pneumatically communicates with an inlet orifice of the mouthpiece;
a vaporizing component received in the vaporizing cavity;
an exhaust cavity of the bifurcated body that pneumatically communicates with an outlet orifice of the mouthpiece, the exhaust cavity pneumatically separated from the vaporizer cavity;
a one-way valve positioned in a selected one of: (i) the exhaust cavity; and (ii) outlet orifice and that: (i) closes in response to inhalation through the inlet orifice of the mouthpiece; and (ii) opens in response to exhalation through the outlet orifice of the mouthpiece; and
an air filter received in the exhaust cavity.

2. The vapor dispenser of claim 1, wherein the exhaust cavity comprises an attachable filter body containing the air filter.

3. The vapor dispenser of claim 1, wherein:
the exhaust cavity comprises a filter door that receives the air filter and comprises: (i) a proximal air conduit that directs exhalation to one side of the air filter; and (ii) a vent that receives exhalation from a second side of the air filter.

4. The vapor dispenser of claim 3, wherein the air filter comprises a cylindrical air filter.

5. The vapor dispenser of claim 3, wherein the air filter comprises a planar air filter.

6. The vapor dispenser of claim 1, wherein the vaporizing component vaporizes a selected one of: (i) cannabis flowers; and (ii) cannabis oil.

7. The vapor dispenser of claim 1, further comprising a cartridge containing a vaporizing material and that is insertable into the vaporizer device.

8. The vapor dispenser of claim 7, wherein vaporizer device comprises a rotatable barrel having at least one cylinder bore to receive the cartridge.
